(19) United States
(12) Patent Application Publication
(10) Pub. No.: US 2014/0377814 A1
(43) Pub. Date: Dec. 25, 2014

(54) Method For The Fixation Or Conversion Of High-Pressure Carbon Dioxide Using Barophilic Sulfur-Oxidizing Chemolithoautotrophs

(71) Applicant: Industry-Academic Cooperation Foundation, Yonsei University, Seoul, SO (KR)

(72) Inventors: Chang Ha Lee, Seoul (KR); Ik Sung Ahn, Seoul (KR); Hyuk Sung Kwon, Uijeongbu-Si (KR); Jae Hyuk Lee, Seoul (KR)

(21) Appl. No.: 14/376,510

(22) PCT Filed: Sep. 20, 2012
(86) PCT No.: PCT/KR2012/007519

§ 371 Date: Aug. 4, 2014

(30) Foreign Application Priority Data
Feb. 7, 2012 (KR) 10-2012-0012521

(51) Int. Cl. C12P 7/64; C12P 7/46; C12P 7/56; C12P 13/22; C12P 13/06; C12P 13/20; C12P 13/14; C12N 1/20; C12P 13/08

(52) U.S. Cl. CPC C12P 7/6409; C12N 1/20; C12P 7/46; C12P 7/56; C12P 13/08; C12P 13/06; C12P 13/20; C12P 13/14; C12P 13/225; C12P 13/222
USPC 435/108; 435/266; 435/289.1; 435/106; 435/136; 435/134; 435/145; 435/139; 435/115; 435/116; 435/109; 435/110

(57) ABSTRACT

The present invention relates to a method for biologically treating carbon dioxide using the sulfur-oxidizing chemolithoautotroph Sulfurovum lithotrophicum 42BKT. The method of the present invention may enable carbon dioxide to be fixed or converted in high-concentration and high-pressure conditions which do not allow the biological photosynthetic conversion of microalgae or the like, and may exhibit high efficiency in the fixation of carbon dioxide as compared to existing methods for biologically treating carbon dioxide using microalgae. Further, the method of the present invention may use a gas mixture without a process of separating nitrogen and other gases, thus simplifying the process of the fixation or conversion of carbon diode. Therefore, according to the present invention, high-pressure carbon dioxide can be treated in an efficient and safe manner by means of the biological conversion of the high-pressure carbon dioxide, and biomass having a high added value can be obtained

### TECHNICAL FIELD

The present invention relates to a method of fixing or converting high-pressure carbon dioxide into a higher value-added biomass using a barophilic sulfur-oxidizing chemolithoautotroph.

### BACKGROUND ART

Carbon dioxide capture and storage (CCS) refers to technology of collecting artificially emitted carbon dioxide, transporting the carbon dioxide to a site isolated from the atmosphere and storing the carbon dioxide. Carbon dioxide capture technology known so far has been developed to separate from an emitted gas a mixture of carbon dioxide with a high purity suitable for storage, and includes an absorption method, a membrane separation method, an adsorption method, and the like. However, such capture technology requires a large-scale processing device and energy, and thus accounts for 50 to 70% of the total estimated cost of the CCS technology. For example, when the best capture technology known so far is applied to a nitrogen/carbon dioxide mixture emitted from a thermoelectric power plant, an increase in exiting electric charges by 15 to 25% tends to be required in all countries.

Also, a storage place in which carbon dioxide can be stably stored so far includes geological strata which have existed stably for several hundred years such as sites in which crude oil has been buried. Technology of storing carbon dioxide in a geological stratum was first proposed in the 1970s, and was intensively studied up to the early 1990s. Many reports were published thereafter (Koide et al., 1992; van der Meer, 1992; Gunter et al., 1993; Holloway and Savage, 1993; Bachu et al., 1994; Korbol and Kaddour, 1994). Statoil and their partner Sleipner launched a large-scale storage project on gas reservoirs in the North Sea for the first time.

The needs for treatment of stored carbon dioxide are mainly divided into three categories. The first category is the leakage of carbon dioxide. Carbon dioxide buried underground is not completely isolated from the atmosphere since it may be emitted into the air after being fixed under the ground for several hundred years. Also, there is also a risk of carbon dioxide which has been buried underground leaking due to subsequent crustal movements of the earth. As a result, sites required to store carbon dioxide should be carefully determined when they are selected, and adequate measures should be provided after storage of carbon dioxide. When a large amount of stored carbon dioxide is emitted into the air, it can have a fatal influence on the global environment. In the year 1980, explosive eruption of high-concentration carbon dioxide at lakes Nyos and Monoun in the Republic of Cameroon took the lives of many animals as well as 2,000 people living near the lakes. The second category is the physicochemical property of carbon dioxide. Sea water becomes acidic when carbon dioxide is dissolved in the sea. The pH level of the sea decreased by 0.1 while the concentration of carbon dioxide in the air changed from 280 ppm to 380 ppm in the year 2004. Since the concentration of carbon dioxide is expected to increase in the future, the pH of the sea is expected to further decrease. It is apparent that such a continuous decrease in pH will result in the destruction of marine ecosystems. Also, even when high-pressure carbon dioxide is buried underground, an underground ecosystem and underground water may be contaminated in the event of leakage due to the acidity of the high-pressure carbon dioxide. The third category is the limitation of storage spaces. As described above, marine reservoirs are not suitable as storage places due to the risk of destruction of marine ecosystems. The essential conditions for geologic storage are a stable foundational structure having no risk of leakage, and regions around which no humans live. Regions satisfying all of these conditions are limited.

The technologies of fixing and converting carbon dioxide, which are currently being developed, include a microalgae culture method and a precipitation method. The chemical precipitation method converts carbon dioxide into CaCO₃ or MgCO₃, which are of very poor economic value. It is not accepted as an ultimate method of treating carbon dioxide due to the capture/transportation/reaction costs and reaction rate. Although the microalgae culture method has an advantage in that carbon dioxide can be converted into useful lipids, it has problems in that it requires a light source, only low-pressure (1 atm or less) carbon dioxide may be used, and it is difficult to apply practically to conversion of some of the carbon dioxide being generated on the Earth.

The present inventors have recognized the limitations of such conventional capture, storage and conversion of carbon dioxide, and found a solution to these problems. As a result, the present inventors developed a method of fixing or converting carbon dioxide using a barophilic sulfur-oxidizing chemolithoautotroph which can use high-pressure carbon dioxide as a carbon source, and filed Korean Patent Application No. 2009-0132548 (Patent Publication No. 10-2011-0075969).

### DISCLOSURE

### Technical Problem

The present invention is directed to providing a method of converting high-pressure carbon dioxide into a higher value-added biomass by promoting an environment suitable for growth of a barophilic sulfur-oxidizing chemolithoautotroph which can use high-pressure carbon dioxide as a carbon source.

### Technical Solution

Sulfurovum lithotrophicum 42BKT (September 2004; 54 (Pt5): 1477-82, BAA-797T=JCM 12117T) used in the method of fixing or converting carbon dioxide according to the present invention was reported as a barophilic sulfur-oxidizing chemolithoautotroph for assimilating carbon dioxide, and its characteristics are described below.

TABLE 1

| | Sulfurovum lithotrophicum |
|---|---|
| Characteristics | 42BKT |
| Shape | |
| Shape of microorganisms | Coccoid oval |
| Mobility | — |
| Growth conditions | |
| Temperature range (° C.) | 10 to 40 |
| Optimum temperature (° C.) | 28 to 30 |
| Doubling time (h) at optimum temperature | 1.5 |
| pH range | 5.0 to 9.0 |
| Optimum pH | 6.7 |
| NaCl requirements | + |
| Peak O₂ concentration (%) | 5 |
| Anaerobic growth | — |
| Sulfur supply | S₂O₃²⁻, S²⁻ |
| Energy metabolism | |
| Electron donor | S₂O₃²⁻, S²⁻ |
| Electron acceptor | NO₃⁻, O₂ |
| Carbon assimilation pathway | rTCA |

The present inventors have continuously researched methods of fixing or converting carbon dioxide using a barophilic sulfur-oxidizing chemolithoautotroph, and found that conditions used to culture Sulfurovum lithotrophicum 42BKT are very important. Especially, among the culture conditions, a change in pH may hinder the growth of Sulfurovum lithotrophicum 42BKT. The change in pH during fixation and conversion of carbon dioxide upon cell growth is caused by various factors, which include: i) reduction in carbon dioxide (carbonic acid), ii) reduction of nitrates, iii) conversion of thiosulfate into sulfates, iv) generation of organic acids, and the like.

As a method to compensate for the decrease in pH caused by the introduction of carbon dioxide, the present inventors used strong alkalis such as NaOH at the beginning of the research. An initial pH level was corrected through introduction of carbon dioxide before cell culture. This was because it was easy to use strong alkalis such as NaOH. However, it was found that the addition of strong alkalis was not useful in compensating the decrease in pH occurring during the cell growth. When NaOH was added, the pH level was adjusted instantaneously, but the dissolution of an excessive carbon dioxide took place in a solution under the same pressure condition due to the presence of NaOH. Also the biological conversion reaction of carbon dioxide did not take place well since the strong alkali came in contact with cells instantaneously.

Therefore, one aspect of the present invention provides a novel optimal culture condition at which a sulfur-oxidizing chemolithoautotroph, Sulfurovum lithotrophicum 42BKT, fixes or converts high-pressure carbon dioxide.

Another aspect of the present invention provides a method of fixing or converting carbon dioxide, which includes culturing Sulfurovum lithotrophicum 42BKT in a medium including a zwitterionic compound or a salt thereof, as a buffering agent, and a sulfur component to fix and convert carbon dioxide. When the zwitterionic compound or salt thereof, such as PIPES, is used as a buffering agent from the very beginning, the zwitterionic compound or salt thereof serves to continuously maintain pH during fixation/conversion of carbon dioxide.

In the present invention, the zwitterionic compound or salt thereof plays an important role in adjusting the pH of the medium containing a high concentration of carbon dioxide. In previous studies, the present inventors simply adjusted the pH using only a strong base such as NaOH, but have since recognized that the addition of the strong base does not guarantee to adjust the pH of the medium in which a high concentration of carbon dioxide is dissolved and to form an environment for the growth of Sulfurovum lithotrophicum 42BKT. As a result, they have made efforts to screen a buffering agent suitable for the growth of Sulfurovum lithotrophicum 42BKT and adjustment of the pH, and found that the zwitterionic compound or salt thereof provides an optimal culture condition.

For example, the zwitterionic compound may include piperazine-N,N′-bis(2-ethanesulfonic acid) (PIPES), 4-(2-hydroxyethyl)-1-piperazineethanesulfonic acid (HEPES), 2-(N-morpholino)ethanesulfonic acid (MES), 2-[[1,3-dihydroxy-2-(hydroxymethyl)propan-2-yl]amino]ethanesulfonic acid (TES), 3-[4-(2-hydroxyethyl)-1-piperazinyl]propanesulfonic acid (HEPPS), N-cyclohexyl-3-aminopropanesulfonic acid (CAPS), 3-(N-morpholino)propanesulfonic acid (MOPS), N-cyclohexyl-2-aminoethanesulfonic acid (CHES), N-2-acetamidoiminodiacetic acid (ADA), N-2-acetamido-2-aminoethanesulfonic acid (ACES), N-[tris(hydroxymethyl)methyl]glycine (Tricine), N,N-bis(2-hydroxyethyl)glycine (Bicine), and N-tris(hydroxymethyl)methyl-3-aminopropanesulfonic acid (TAPS), but the present invention is not limited thereto.

According to one exemplary embodiment, the zwitterionic compound or salt thereof may be a compound or a salt thereof represented by the following Formula 1:

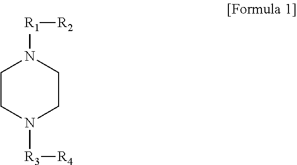

In Formula 1,

R₁ and R₃ each independently represent an alkylene group having 1 to 4 carbon atoms, and

R₂ and R₄ each independently represent a hydroxyl group or a sulfonic acid (—SO₃H) group.

According to one exemplary embodiment, the compound or salt thereof of Formula 1 may be piperazine-N,N′-bis(2-ethanesulfonic acid) (PIPES), 4-(2-hydroxyethyl)-1-piperazineethanesulfonic acid (HEPES), or a monosodium salt thereof, disodium salt thereof or mixture thereof.

In the present invention, the pH of the medium in which Sulfurovum lithotrophicum 42BKT grows may be in a range of 5 to 9, but the present invention is not limited thereto.

Since Sulfurovum lithotrophicum 42BKT is a sulfur-oxidizing chemolithoautotroph, Sulfurovum lithotrophicum 42BKT uses sulfur as an essential nutrient source. Therefore, sulfur is essentially included in the medium used in the method according to the present invention. The sulfur may be included in the medium as sulfur, sodium sulfide (Na₂S), sodium thiosulfate (Na₂S₂O₃), or a mixture thereof, but the present invention is not limited thereto.

According to one exemplary embodiment of the present invention, the sulfur may be continuously supplied into the medium to constantly fix and convert carbon dioxide.

Since Sulfurovum lithotrophicum 42BKT is a microorganism present in the deep sea, the medium may include saline water. The saline water may be at least one selected from the group consisting of sea water, saline water of an unused saline water-saturated reservoir rock, and synthetic sea water, but the present invention is not limited thereto. The saline water included in the medium may be selected among the sea water, the saline water of the unused saline water-saturated reservoir rock and the synthetic sea water depending on the position of a carbon dioxide storage place. Components of the saline water are well known in the related art. For example, the saline water may include NaCl, NaNO₃, NaS₂O₃, NH₄Cl, K₂HPO₄, and the like.

According to one exemplary embodiment, the medium may further include one of a trace mineral supplement and a trace vitamin supplement. For example, the trace mineral supplement or the trace vitamin supplement may be optionally used in the synthetic sea water since the natural sea water contains components deficient in the synthetic sea water.

As seen in examples below, Sulfurovum lithotrophicum 42BKT may grow at a high pressure unlike microorganisms or algae which have previously been used to biologically treat carbon dioxide. A total gas pressure containing carbon dioxide in the carbon dioxide storage place may be in the range from 1 to 200 atmospheres (atm), but the present invention is not limited thereto.

Exhaust carbon dioxide is discharged in the form of a mixture. In this case, a large amount of energy is required to capture the exhaust carbon dioxide. Also, high-purity carbon dioxide should be captured to geologically store carbon dioxide, but a change in a geological environment may be caused due to acidity of such high-purity carbon dioxide. Unlike conventional microorganisms or algae, which are killed by SO₂ or NO introduced when CO₂ is captured from commercial exhaust gases, Sulfurovum lithotrophicum 42BKT is tolerant to SO₂ or NO. Therefore, it is possible to perform stable biological treatment of carbon dioxide. In the following examples, it is shown that carbon dioxide can be fixed and converted by Sulfurovum lithotrophicum 42BKT without the separation and capture of carbon dioxide from the mixture of nitrogen/carbon dioxide, which is an emission gas in a thermoelectric power plant. Therefore, in the method of the present invention, the carbon dioxide in the carbon dioxide storage place may be present as pure carbon dioxide or a carbon dioxide mixture.

According to the present invention, Sulfurovum lithotrophicum 42BKT shows excellent tolerance to high-concentration carbon dioxide. As a result, the high-concentration carbon dioxide may be used to culture Sulfurovum lithotrophicum 42BKT without any dilution, thereby simplifying a process for biologically treating carbon dioxide. According to one exemplary embodiment, the partial pressure of the carbon dioxide provided as the carbon source in the method of fixing or converting carbon dioxide may be in a range of 0.1 to 50 atm.

Meanwhile, the temperature required to fix or convert carbon dioxide may be in a range of 4 to 40° C. In the method according to the present invention, microorganisms using carbon dioxide as the carbon source may grow within this temperature range.

Still another aspect of the present invention provides a composition for fixing or converting carbon dioxide, which includes a zwitterionic compound or a salt thereof; a sulfur component; and Sulfurovum lithotrophicum 42BKT. Descriptions of the zwitterionic compound and the medium have been provided above.

Still another aspect of the present invention provides a device for fixing or converting carbon dioxide, which includes a carbon dioxide storage place containing the composition for fixing or converting carbon dioxide, and an injection port through which a gas including carbon dioxide is injected into the storage place.

According to one exemplary embodiment, the carbon dioxide storage place in which the carbon dioxide is fixed or converted may be a depleted oil or gas reservoir, a deep unused saline water-saturated reservoir rock, a deep unminable coal seam, a basalt, an oil shale, a cavity, or a geologic storage place such as an underground reactor. This geologic storage place may be properly used to store and treat high-pressure carbon dioxide.

According to another exemplary embodiment, the carbon dioxide storage place may be a ground reactor. When a reactor is installed on the ground, captured carbon dioxide may be directly treated in the reactor in a site in which carbon dioxide is generated. As a result, the transportation costs may be saved, and it is more inexpensive and safe than underground storage of carbon dioxide. In addition, a mixture of carbon dioxide may be directly treated without using a separator for separating a mixture of carbon dioxide to remove contaminants such as sulfur, nitrogen compounds, dusts, hydrocarbons, and the like. The method according to the present invention may be used to convert carbon dioxide in the ground reactor, may be used in many countries having no geologic storage places, and may increase a pressure in a ground reactor in order to reduce the volume of the ground reactor, thereby realizing excellent industrial applicability.

Yet another aspect of the present invention provides a method of preparing an organic material, which is at least one selected from the group consisting of an amino acid, an organic acid and a fatty acid, which includes culturing Sulfurovum lithotrophicum 42BKT in the presence of carbon dioxide in a medium including a zwitterionic compound or a salt thereof, as a buffering agent, and a sulfur component.

As seen in examples below, it was found that the barophilic sulfur-oxidizing chemolithoautotroph used highly-concentrated carbon dioxide for its own growth at a high pressure, or converted into an organic material such as an organic acid such as succinic acid, lactic acid, or the like; an amino acid such as alanine, valine, leucine, isoleucine, aspartic acid, pyroglutamic acid, glutamic acid, phenylalanine, lysine, tyrosine, or the like; or a fatty acid such as vaccenic acid, palmitoleic acid, or the like.

According to one exemplary embodiment, the organic material may be at least one selected from the group consisting of succinic acid, lactic acid, alanine, valine, leucine, isoleucine, aspartic acid, pyroglutamic acid, glutamic acid, phenylalanine, lysine and tyrosine, vaccenic acid, and palmitoleic acid.

The grown sulfur-oxidizing chemolithoautotroph may be used as a biomass, and the synthesized organic materials may be used as starting materials in white biotechnology by which current chemicals depending on fossil fuels can be replaced. Therefore, the method of fixing and converting carbon dioxide according to the present invention has advantages in that it can be used to biologically treat carbon dioxide and recycle carbon dioxide, which has been disposed of as a waste, as a new resource.

### Effect of the Invention

The method for fixing or converting carbon dioxide using the Sulfurovum lithotrophicum 42BKT can be useful in fixing or converting carbon dioxide even under high-concentration and high-pressure carbon dioxide conditions in which carbon dioxide has not been fixed or converted in photosynthetic conversion of microalgae, and exhibiting higher efficiency in fixation of carbon dioxide than a method for biologically treating carbon dioxide using microalgae, compared to conventional methods for biologically treating carbon dioxide using microalgae. Further, in the method, a gas mixture can be used without a process of separating nitrogen and other gases, which has been required for biological treatment of carbon dioxide, thereby simplifying a process of fixing or converting carbon dioxide. Therefore, the method can be useful in enabling efficient and safe treatment of carbon dioxide by means of biological conversion of high-pressure carbon dioxide and simultaneously obtaining a higher value-added biomass.

### BRIEF DESCRIPTION OF THE DRAWINGS

FIG. 1 shows gas chromatogram of a gas sample from the biological treatment of carbon dioxide using Sulfurovum lithotrophicum 42BKT.

FIGS. 2 to 4 show GC-MS analysis results on a frech medium (FIG. 2) and a medium after the culture (FIG. 3) (i.e., a medium from which microorganisms are removed after biological treatment) in an experiment for biological treatment of carbon dioxide using Sulfurovum lithotrophicum 42BKT; and the inside of Sulfurovum lithotrophicum 42BKT (FIG. 4);

FIGS. 5 and 6 are images showing changes in cell concentrations in an early stage (FIG. 5) and a late stage (FIG. 6) of an experiment on Sulfurovum lithotrophicum 42BKT used in the experiment for biological treatment of carbon dioxide.

MODE FOR INVENTION Advantages and characteristics of the present invention, and a method of achieving them will become clear with reference to the following Examples as mentioned below in detail. However, the present invention is not limited to the following Examples, and various types of the present invention will be implemented in various manners. The Examples are disclosed merely to provide a complete description of the present invention and to provide complete understanding of the present invention to those skilled in the art to which the present invention belongs, and the present invention is only defined by the appended claims.

### EXAMPLES

1. Preparation of Medium

A medium was prepared as listed in Table 2 (components of the medium for growth of Sulfurovum lithotrophicum 42BKT) to fix and convert carbon dioxide using Sulfurovum lithotrophicum 42BKT.

TABLE 2

| | | |
|---|---|---|
| MJ (—N) synthetic sea water | 1.0 | L |
| NH₄Cl | 0.25 | g |
| Na₂S₂O₃•5H₂O | 1.5 | g |
| NaNO₃ | 2.0 | g |
| Trace mineral | 10 | ml |
| Trace vitamin | 1.0 | ml |
| PIPES sodium salt | 16.2 | g |
| PIPES disodium salt | 17.3 | g |

2. Treatment and Conversion of High-Pressure Carbon Dioxide Using Sulfurovum lithotrophicum 42BKT

1) Measurement of Dissolution of Carbon Dioxide in Microorganism-Free Medium

i. 160 ml of a fresh medium was put into a sterile high-pressure reactor, and an inner space of the reactor was replaced for approximately 60 seconds with a gas mixture in which nitrogen and carbon dioxide were mixed at a ratio of 8:2. In this case, the reactor was maintained at a temperature of 28 to 30° C.

ii. A pressure in the reactor was slowly increased to 10 atm using the gas mixture in which nitrogen and carbon dioxide were mixed at a ratio of 8:2.

iii. Thereafter, changes in pressure and pH over time were observed.

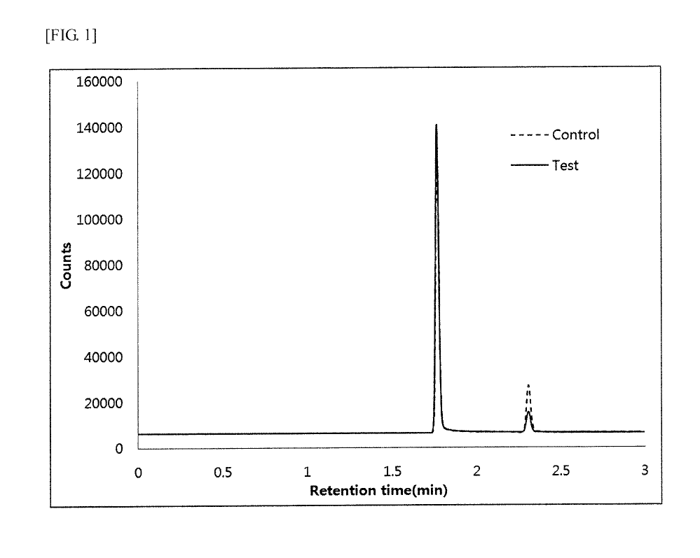

iv. After a lapse of sufficient time, a gas sample was taken out, and saturation dissolution of carbon dioxide was measured using gas chromatography. The gas chromatography results are shown by a dotted line (control) in FIG. 1.

2) Measurement of Treatment of Carbon Dioxide in Microorganism-Containing Medium

i. 4 ml of a Sulfurovum lithotrophicum 42BKT culture was added to 160 ml of a fresh medium, which was put into a sterile high-pressure reactor, and an inner space of the reactor was replaced for approximately 60 seconds with a gas gas mixture in which nitrogen and carbon dioxide were mixed at a ratio of 8:2. In this case, the reactor was maintained at a temperature of 28 to 30° C.

ii. A pressure in the reactor was slowly increased to 10 atm using the gas mixture in which nitrogen and carbon dioxide were mixed at a ratio of 8:2.

iii. Thereafter, changes in pressure and pH over time were observed.

iv. After a lapse of sufficient time, the pressure in the reactor was lowered to a level of atmospheric pressure when the turbidity of the culture broth increased.

v. A gas sample was taken out, and saturation dissolution of carbon dioxide was measured using gas chromatography. The gas chromatography results are shown by a solid line (experimental group) in FIG. 1.

As shown in FIG. 1, it could be seen that carbon dioxide was treated by the activities of Sulfurovum lithotrophicum 42BKT. Unlike the technology of treating carbon dioxide at a low concentration under the atmospheric pressure conditions as known in the related art, this indicates that carbon dioxide is treated at a high pressure and a high concentration.

Through this treatment, the carbon dioxide was converted into a biomass. Table 3 lists a concentration of a microorganism measured after inoculation of the microorganism into an early medium, a concentration of a microorganism cultured in a late medium, an amount of grown microorganism, a dry weight of the microorganism, and a ratio of organic carbon in the dry weight of the microorganism.

TABLE 3

| | | |
|---|---|---|
| Concentration of microorganism in | (6.1 ± 0.1) × 10⁶ | cells/ml |
| early medium | | |
| Concentration of microorganism | (2.5 ± 0.05) × 10⁸ | cells/ml |
| cultured in late medium | | |
| Amount of grown microorganism | (2.435 ± 0.045) × 10¹¹ | cells/L |
| Dry weight of microorganism | (1.6 ± 0.05) × 10⁻¹³ | g/cells |

| | |
|---|---|
| Ratio of organic carbon | 45.95 ± 0.71% |

The biomass was calculated using the data listed in Table 3. As a result, a conversion rate of carbon dioxide into the biomass was 67.33+1.31 g CO²/g cells·L·day when the conversion of carbon dioxide was performed using Sulfurovum lithotrophicum 42BKT.

In particular, when carbon dioxide was present at a state of high concentration or high pressure, the carbon dioxide was not treated using microalgae. However, the method according to the present invention has great significance since the size of a ground reactor can be reduced in proportion to the pressure applied when the ground reactor is used for treatment of the high-pressure carbon dioxide.

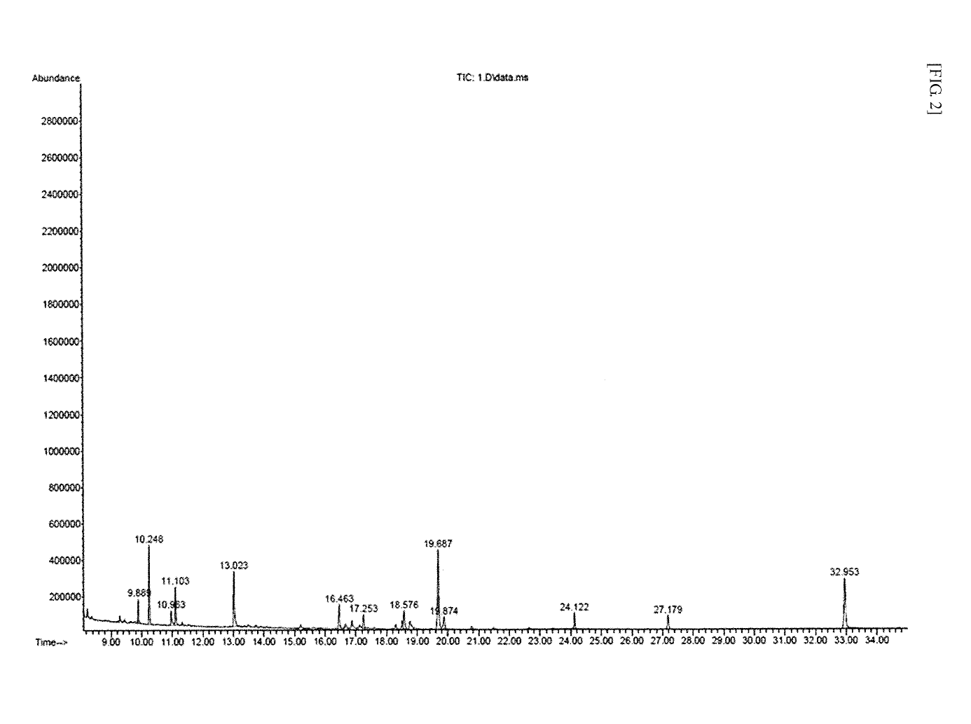

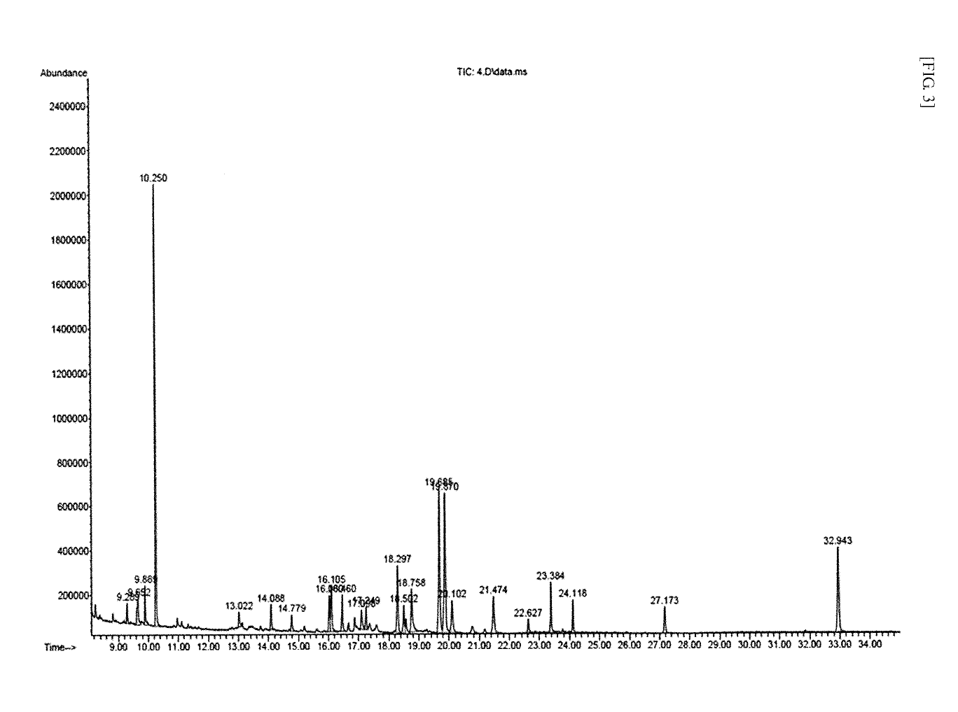

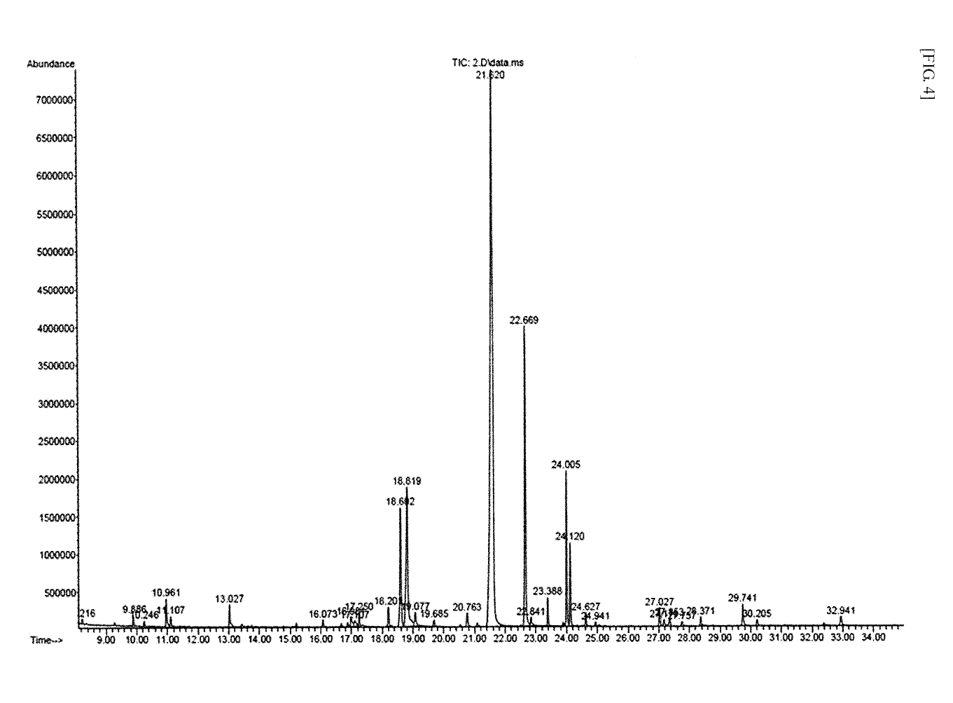

Furthermore, GC-MS analyses were performed on an early medium (FIG. 2) and a late medium (FIG. 3) (i.e., a medium from which microorganisms were removed after biological treatment) in an experiment for biological treatment of carbon dioxide using Sulfurovum lithotrophicum 42BKT; and the inside of Sulfurovum lithotrophicum 42BKT (FIG. 4). As shown in FIGS. 2 to 4 and listed in Table 4, it was revealed that the carbon dioxide biologically treated in the above-described experiment was used for growth of Sulfurovum lithotrophicum 42BKT, or converted into succinic acid, lactic acid, alanine, valine, leucine, isoleucine, aspartic acid, pyroglutamic acid, glutamic acid, phenylalanine, lysine, tyrosine, vaccenic acid, and palmitoleic acid. Table 4 lists the organic materials, which are included in the late medium and the inside of the microorganism, and retention times thereof

TABLE 4

| No. | Retention time (min) | Peak name |
|---|---|---|
| 1 | 10.96 | Succinic acid |
| 2 | 11.10 | Lactic acid |
| 3 | 13.02 | Alanine |
| 4 | 14.77 | Valine |
| 5 | 16.08 | Leucine |
| 6 | 16.10 | Isoleucine |
| 7 | 18.55 | Aspartic acid |
| 8 | 18.80 | Pyroglutamic acid |
| 9 | 21.62, 22.66 | Glutamic acid |
| 10 | 23.38 | Phenylalanine |
| 11 | 24.00 | Palmitoleic acid |
| 12 | 27.02 | Vaccenic acid |
| 13 | 29.74 | Lysine |
| 14 | 32.94 | Tyrosine |

As described above, the organic materials (including succinic acid) produced by converting carbon dioxide using Sulfurovum lithotrophicum 42BKT may be used as the starting material for development of various chemical materials. Therefore, the method of fixing or converting carbon dioxide according to the present invention may be used to biologically treat carbon dioxide and recycle carbon dioxide, which has been disposed of as a waste, as a new resource.

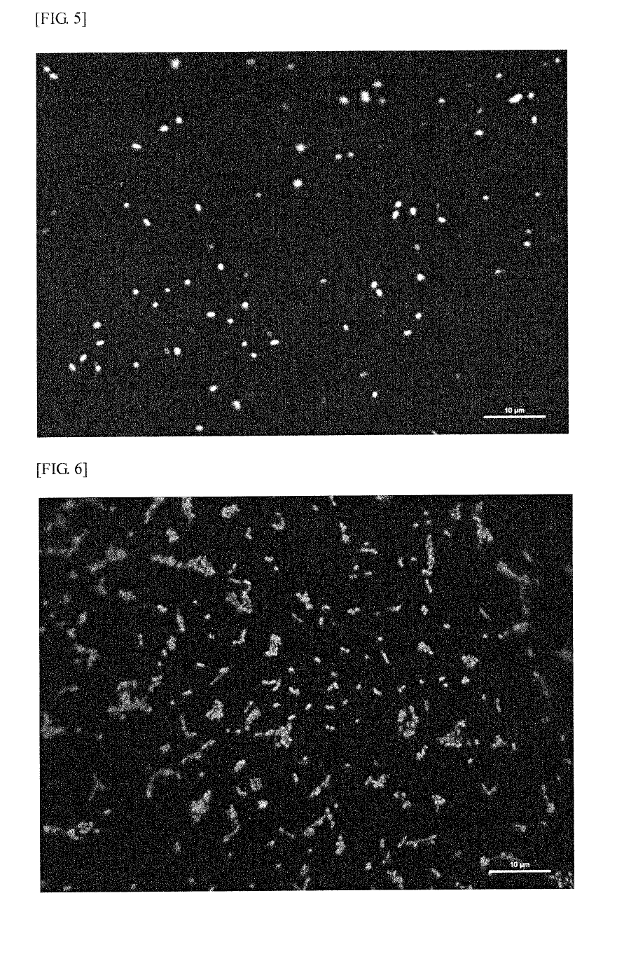

In the early and late stages of an experiment for biological treatment of carbon dioxide, Sulfurovum lithotrophicum 42BKT was stained with acridine orange, and observed under a fluorescence microscope (Filtering volume: 0.005 μl, Magnification×100, Scalebar: 10 μm). FIGS. 5 and 6 are images showing changes in cell concentration in the early stage (FIG. 5) and the late stage (FIG. 6) of the experiment on Sulfurovum lithotrophicum 42BKT used in the experiment for biological treatment of carbon dioxide. The grown Sulfurovum lithotrophicum 42BKT may be recycled to fix carbon dioxide or may be used as a biomass.

1. A method of fixing or converting carbon dioxide, comprising:
culturing Sulfurovum lithotrophicum 42BKT in a medium including a zwitterionic compound or a salt thereof, as a buffering agent, and a sulfur component to fix and convert carbon dioxide.

2. The method of claim 1, wherein the zwitterionic compound or salt thereof is a compound or a salt thereof represented by the following Formula 1: 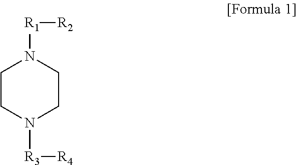
wherein R₁ and R₃ each independently represent an alkylene group having 1 to 4 carbon atoms, and
R₂ and R₄ each independently represent a hydroxyl group or a sulfonic acid (—SO₃H) group.

3. The method of claim 2, wherein the zwitterionic compound or salt thereof of Formula 1 is piperazine-N,N′-bis(2-ethanesulfonic acid) (PIPES), 4-(2-hydroxyethyl)-1-piperazineethanesulfonic acid (HEPES), or a monosodium salt thereof, disodium salt thereof or mixture thereof.

4. The method of claim 1, wherein the medium has a pH of 5 to 9.

5. The method of claim 1, wherein the sulfur component is sulfur, sodium sulfide (Na₂S), sodium thiosulfate (Na₂S₂O₃), or a mixture thereof, and is included in the medium.

6. The method of claim 1, wherein the sulfur component in the medium is continuously supplied to constantly perform fixation and conversion of carbon dioxide.

7. The method of claim 1, wherein the medium comprises saline water.

8. The method of claim 7, wherein the saline water is at least one selected from the group consisting of sea water, saline water in an unused saline water-saturated reservoir rock, and synthetic sea water.

9. The method of claim 1, wherein the medium further comprises at least one of a trace mineral supplement and a trace vitamin supplement.

10. The method of claim 1, wherein a gas containing the carbon dioxide has a total pressure of 1 to 200 atmospheres (atm).

11. The method of claim 1, wherein the carbon dioxide is present as pure carbon dioxide or a mixture of carbon dioxide and another gas.

12. The method of claim 1, wherein the carbon dioxide has a partial pressure of 0.1 to 50 atm.

13. The method of claim 1, wherein the temperature required to fix and convert carbon dioxide is in a range of 4 to 40° C.

14.-20. (canceled)

21. A device for fixing or converting carbon dioxide, comprising:
a carbon dioxide storage place containing a composition comprising a zwitterionic compound or a salt thereof; a sulfur component; and Sulfurovum lithotrophicum 42BKT; and
an injection port through which a gas comprising carbon dioxide is injected into the storage place.

22. The device of claim 21, wherein the carbon dioxide reservoir is a depleted oil or gas reservoir, a deep unused saline water-saturated reservoir rock, a deep unminable coal seam, a basalt, an oil shale, a cavity, an underground reactor, or a ground reactor.

23. A method of preparing an organic material, which is at least one selected from the group consisting of an amino acid, an organic acid and a fatty acid, comprising:
culturing Sulfurovum lithotrophicum 42BKT in the presence of carbon dioxide in a medium including a zwitterionic compound or a salt thereof as a buffering agent, and a sulfur component.

24. The method of claim 23, wherein the organic material is at least one selected from the group consisting of succinic acid, lactic acid, alanine, valine, leucine, isoleucine, aspartic acid, pyroglutamic acid, glutamic acid, phenylalanine, lysine and tyrosine, vaccenic acid, and palmitoleic acid.
